(19) United States
(12) Patent Application Publication
(10) Pub. No.: US 2010/0184977 A1
(43) Pub. Date: Jul. 22, 2010

(54) Nickel or Iron Catalysed Carbon-Carbon Coupling Reaction of Arylenes, Alkenes and Alkines

(76) Inventors: Paul knochel, Gauting (DE); Andrei Gavriushin, Germering (DE); Christiane Charlotte Kofink, Munchen (DE); Georg Manolikakes, Munchen (DE)

Correspondence Address: LANXESS CORPORATION, 111 RIDC PARK WEST DRIVE, PITTSBURGH, PA, 15275-1112, US

(21) Appl. No.: 11/991,820

(22) PCT Filed: Sep. 12, 2006
(86) PCT No.: PCT/EP2006/066270

§ 371 Date: Mar. 25, 2010

(30) Foreign Application Priority Data
Sep. 12, 2005 (DE) 10 2005043 337.5

(51) Int. Cl. C07D 239/26; C07D 215/04; C07D 401/04; C07D 405/04; C07D 213/30; C07D 209/04; C07C 253/30; C07C 67/30; C07C 45/45; C07C 41/30

(52) U.S. Cl. 544/242; 546/173; 546/276.4; 546/283.4; 546/283.7; 546/339; 548/469; 558/359; 560/59; 568/315; 568/642

(57) ABSTRACT

Organozinc compounds of the type R¹—Ar¹—ZnY (1) can be reacted with different functionalized aryl halides R²—Ar²—X (2) in the presence of catalytic amounts of Ni or Fe in a polar solvent or solvent mixture to form polyfunctional biaryles of the type R¹—Ar¹—Ar²—R² (3). Organozinc compounds of the type (1) can be represented by the transmetallation reaction of functionalized aryl magnesium halides or lithium aryl compounds with e.g. ZnBr₂.

The present invention relates to a method of forming carbon-carbon bonds based on a zinc compound of an aryl, heteroaryl, alkene or alkyne and an aryl, heteroaryl, alkene or alkyne compound with a suitable leaving group.

### BACKGROUND OF THE INVENTION

Transition metal-catalysed cross coupling reactions are very efficient reactions to form C—C bonds, in particular between Csp² centres, where typical S_{N}2 substitutions cannot be conducted.^{[1]} The aryl-aryl cross coupling is one of the most important methods of forming carbon-carbon bonds. Many of the aromates and in particular of the heteroaromates obtained therewith are of great importance not only for the agronomical and pharmaceutical industry but also for materials sciences. Here, mostly reliable palladium(0) catalysts,^{[1,2]} primarily in the presence of a respective ligand, such as for example sterically hindered phosphines, are used.^{[3]} The palladium-phosphine complexes are usually used in an amount of 1-3 mole percent. As, however, not only palladium is expensive but also the respective phosphine ligands, there is a need of favourable and highly effective catalysts.

According to the work of Kochi^{[4]} iron-catalysed cross coupling reactions have recently been ascertained very intensely with regard to their efficiency in cross coupling reactions.^{[5]} Although very efficient cross coupling reactions between a number of alkyl magnesium reagents and aryl halides or aryl sulfonates have been achieved, the catalysed cross coupling of two aryl groups still remains problematic, due to the considerable homo coupling reaction of the aryl magnesium species and no synthetic way of solving this problem has been found up to now.^{[5,6]} But at the same time the dehalogenation of the aryl halide takes place as well.

The object of the present invention is thus to provide a simple method of forming carbon-carbon bonds between aryls, alkenes and alkynes in a targeted way in high yields and at low costs.

This object is fulfilled according to the present invention by claim 1. Preferred embodiments are illustrated in the depending claims.

### SUMMARY OF THE INVENTION

The inventors directed their attention to other organometallic families and found that organozinc compounds of the type R¹—Ar¹—ZnY (1) react with different functionalized aryl halides R²—Ar²—X (2) in the presence of catalytic amounts of Ni or Fe in a polar solvent or solvent mixture and result in the formation of polyfunctional biaryls of the type (3).

R¹—Ar¹—Ar²—R² (3)

Organozinc compounds of the type (1) may be prepared by the transmetallation reaction of functionalized aryl magnesium halides or lithium aryl compounds with ZnBr₂ for example. Herein and in the following the term aryl is to be understood as aryl, heteroaryl, alkene or alkyne. These compounds may be mono- or polysubstituted. Essential for the invention is the presence of an aryl, alkene or alkyne compound, the chemical reaction starting off at their characteristic aryl, alkene or alkyne features.

A first aspect of the present invention relates to a method for preparing a compound represented by the general formula (3)

R¹—Ar¹—Ar²—R² (3)

by reacting a compound represented by the general formula (1)

R¹—Ar¹—ZnY (1)

with a compound represented by the general formula (2)

R²—Ar²—X (2)

by effect of a Ni or Fe catalyst in a solvent,
wherein
X may be a leaving group suitable for a nucleophilic substitution;
Y may be Cl, Br, I, R¹COO, ½ SO₄, NO₃, R¹SO₃;
R¹ and R² may each represent independently from one another one or more substituents of H; substituted or unsubstituted aryl or heteroaryl, containing one or more heteroatoms; straight-chain, branched or cyclic, substituted or unsubstituted alkyl, alkenyl, alkynyl; or derivatives thereof;
Ar¹ and Ar² may each represent independently from one another an aryl, condensed aryl, heteroaryl or condensed heteroaryl, containing one or more heteroatoms; an alkenyl or alkynyl; or derivatives thereof;

The leaving group X represents a traditionally used leaving group for a nucleophilic substitution. The group referred to as Ar may also be substituted with several substituents conforming to the definition of R¹ or R², if possible.

According to one embodiment of the present invention, the reaction is effected at a temperature between 0° C. and 150° C., preferably between 10° C. and 120° C., even more preferably between 20° C. and 100° C. and most preferably between 25° C. and 80° C.

According to another embodiment, the catalyst comprises a Fe(III) complex, a Fe(III) salt, a Fe(II) complex, a Fe(II) salt or a reduced form of a Fe salt or complex, preferably Fe(acac)₃ or Fe(DBM)₃, wherein iron is coordinated to acetylacetonate (acac) or dibenzoylmethane (DBM).

According to another embodiment, the catalyst comprises a Ni(II) and/or a Ni(0) catalyst, or another reduced form of a Ni(II) salt and/or complex.

According to yet another embodiment, the catalyst represents a complex with aza-heterocycles, polyaza-heterocycles and/or phosphites represented by the general formula (R^{a}O)₂P(O)H, wherein R^{a} is a straight-chain, branched or cyclic, substituted or unsubstituted alkyl, preferably with a chain length of C₁ bis C₁₀, as ligands.

According to another embodiment, X may preferably be I, Br, Cl, OTf, N₂⁺, OSO₂R^{S} or OP(O)(OR^{S})₂, wherein R^{S} is a straight-chain, branched or cyclic, substituted or unsubstituted alkyl, condensed aryl, substituted or unsubstituted aryl or heteroaryl, more preferably I or Br, even more preferably I.

According to another embodiment, the compound (1) is added in a molar ratio of 0.2-5, preferably in a molar ratio of 1-3, even more preferably in a molar ratio of 1.1-2.5 with regard to the molar amount of compound (2).

According to another embodiment, R¹ and R² may each be independently from one another a substituted or unsubstituted C₄-C₂₄ aryl or C₃-C₂₄ heteroaryl, containing one or more heteroatoms such as B, O, N, S, Se, P; a straight-chain or branched, substituted or unsubstituted C₁-C₂₀ alkyl, C₂-C₂₀ alkenyl, C₂-C₂₀ alkynyl; or a substituted or unsubstituted C₃-C₂₀ cycloalkyl; or derivatives thereof.

According to another embodiment, the catalyst is used in a molecular ratio of 0.00001 to

10%, more preferably of 0.001 to 1 mole percent, even more preferably of 0.02 to 0.2 mole percent with regard to the compound represented by the formula (1) or (2).

According to another embodiment, a polar solvent or solvent mixture, preferably an etherial solvent, a dipolar, aprotic solvent or their solvent mixtures, and most preferably a solvent, selected from the group comprising THF, DME, NMP, DMAC and their mixtures, is used as solvent.

This new approach grants economical access (about three times more favourable in comparison with Pd-catalysed reactions) for effecting aryl-aryl cross coupling reactions.

### DETAILED DESCRIPTION OF THE INVENTION

The present invention will be described in more detail in the following.

Unless determined otherwise, the technical and scientific terms used herein shall have the same meaning as understood by those skilled in the art of this invention.

The organozinc compounds used in the cross coupling can easily be prepared by transmetallation reaction of the respective magnesium or lithium organometall compounds (Knochel, P.; Dohle, W.; Gommermann, N.; Kneisel, F. F.; Kopp, F.; Korn, T.; Sapountzis, I.; Vu, V. A. Angew. Chem. Int. Ed. 2003, 42, 4302.) Direct insertion of the zinc is also possible (Rieke R. D. Science 1989, 246, 1260.; Burns, T. P.; Rieke, R. D. J. Org. Chem. 1987, 52, 3674.; Lee, J.; Velarde-Ortiz, R.; Guijarro, A.; Wurst, J. R.; Rieke, R. D. J. Org. Chem. 2000, 65, 5428), as well as by I/Zn displacement reaction (Kneisel, F. F.; Dochnahl, M.; Knochel, P. Angew. Chem. Int. Ed. 2004, 43, 1017.; Gong, L.-Z.; Knochel, P. Synlett 2005, 267). These different approaches allow for easy access to the zinc-organic starting compounds.

### Iron-Catalysed Chemical Reaction

As mentioned above, respective aryl-zinc compounds can easily be obtained by transmetallation of the respective aryl-Grignard compound. A schematic representation of the chemical reaction is shown in scheme 1 below.

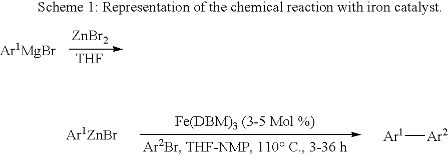

Hereby a cross coupling reaction between the aryl-zinc compound Ar¹ZnBr with an aryl bromide Ar²Br is effected in the presence of iron(III)-tris-dibenzoylmethanate, Fe(DBM)₃, in a solvent mixture such as THF-NMP. The iron catalyst is exemplarily added in 3 to 5 mole percent here and the reaction is effected at a temperature of for example 110° C. ranging from 3 to 36 hours.

Any iron(II) and/or iron(III) salts and/or complexes such as e.g. FeCl₂, FeCl₃, FeBr₂, FeBr₃, Fe(OAc)₂, Fe(OAc)₃ etc. and/or other iron complexes having iron in other oxidation states, also reduced iron complexes, in which the iron has a negative oxidation state, or their mixtures, may be used as iron compounds.

The iron catalyst may preferably be used in an amount ranging from 0.01 to 10 mole percent, more preferably in an amount from 0.1 to 8 mole percent and most preferably from 0.5 to 6 mole percent with regard to one of the reactants (1) or (2).

As can be seen from table 2, the cross coupling products are obtained in good yields. The aryl bromides used exemplarily herein may in turn be substituted. A substitution with fluorine, chlorine, trifluoromethyl or carboethoxy does not hinder the chemical reaction. Heterocyclic aryl compounds, such as e.g. 3-bromopyridine, are amenable for the chemical reaction as well. Zinc compounds, bearing electrophilic groups, such as e.g. an ester group, may also be used for the reaction (see entry c in table 2).

### Nickel-Catalysed Chemical Reaction

The present invention can particularly preferred be effected by effect of nickel catalysts. As already mentioned above, the respective zincorganic compounds are easily accessible in different ways.

Exemplarily a respective chemical reaction of a cross coupling could be represented as follows:

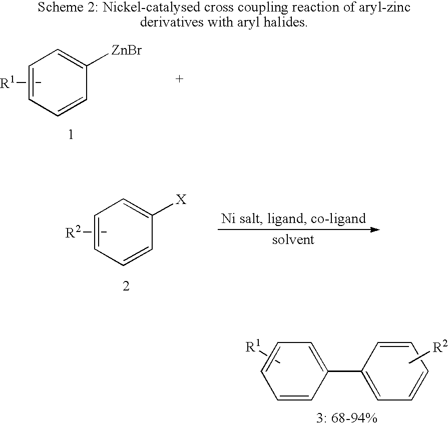

The chemical reaction using nickel as catalyst offers various advantages with regard to the chemical reaction that has been used up to now. The reaction can be effected at significantly lower temperatures ranging between 0 and 100° C. The reaction may therefore also be effected with heat sensitive reactants and products. Furthermore, in the industrial sector no or hardly any additional energy, such as for example heating or irradiation has to be applied.

The used amount of nickel catalyst may also advantageously be extremely low. Hereby preferably used are molecular ratios of 0.00001 to about 10 mole percent, more preferably of 0.001 to 1 mole percent, even more preferably of 0.02 to 0.2 mole percent with regard to one of the reactants (1) or (2). Such low amounts of catalyst do not only represent a cost advantage, but must also be evaluated as advantageous with regard to environmental aspects.

Nickel salts or complexes having the oxidation state II or nickel complexes having the oxidation state (0) may be used as nickel compounds. The complexes Ni(COD)₂, Ni(R¹₃P)₄, Ni((R¹O)₃P)₄ can be mentioned exemplarily as complexes, wherein COD means 1,5-cyclooctadiene and R¹ is defined as above. Nickel salts may for example be selected from the group comprising NiCl₂, NiBr₂, Ni(OAc)₂, Ni(acac)₂, Ni(NO₃)₂, NiSO₄. Using NiCl₂ is particularly preferable.

Phosphites (R^{a}O)₂P(O)H and nitrogen containing heterocycles represented by the following general formulas may be used as complex ligands:

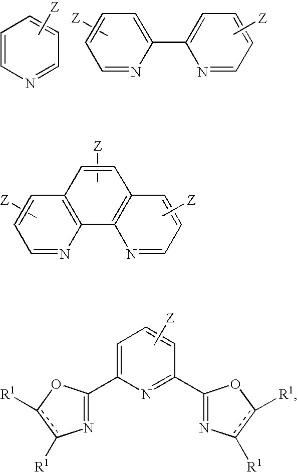

wherein Z=R¹, OR¹, NR¹₂, halide, cyano, annelated substituted and unsubstituted rings, R¹ and R² are as defined above, and wherein R^{a} is a straight-chain, branched or cyclic, substituted or unsubstituted alkyl.

Preferably (MeO)₂P(O)H, (EtO)₂P(O)H, (n-PrO)₂P(O)H, (n-BuO)₂P(O)H, (i-BuO)₂P(O)H are used as phosphites. Diethylphosphite, (EtO)₂P(O)H, is particularly preferred herein. As a nitrogen containing heterocycle, 4-dimethylaminopyridine (DMAP) turned out to be advantageous. The various complex ligands may be used singularily or in combination. The combination of (EtO)₂P(O)H and DMAP turned out to be particularly advantageous herein.

The complex ligands are preferably used in an amount of 0.001 to 5 mole percent, more preferably in an amount of 0.01 to 1 mole percent, even more preferably of 0.1 to 0.5 mole percent and most preferably in an amount of 0.2 mole percent with regard to one of the reactants (1) or (2). Also with regard to the low amount of catalyst metal, low amounts of used complex ligands represent a cost advantage and lower contamination of the environment.

In general etherial solvents or dipolar aprotic solvents or their mixtures may be used as solvents. Examples of such solvents include tetrahydrofuran (THF), dimethylimidazolidnone (DMI), N,N′-dimethylpropyleneurea (DMPU) or 1,2-dimethoxyethane (DME) and N-substituted pyrrolidones, such as e.g. N-ethylpyrrolidone (NEP), N-methylpyrrolidone (NMP), N-2-methoxyethylpyrrolidinone and N,N′-dimethylimidazolidine-2-one, however, they are not limited to them. Furthermore, N,N-dimethylacetamide (DMAC) may be used. Particularly suitable are mixtures of etherial solvents and nitrogen containing solvents. Preferred mixing ratios hereby range between 20:1 and 1:20 of etherial solvent to nitrogen containing solvent.

The advantageous properties of the invention will now be exemplarily illustrated by means of some examples. These examples shall, however, not be interpreted as limitating the invention.

### EXAMPLES

Unless indicated otherwise, all reactions were conducted by stirring magnetically and in case of air-sensitive or hygroscopic compounds in annealed glass gadgets under argon as inert gas. Syringes were used to transfer the reagents and the solvents were rinsed with argon before their use. The reactions were controlled by gas chromatography (GC and GC-MS) or thin-layer chromatography. Solutions of organo magnesium compounds were prepared by reacting magnesium with aryl bromides in THF, if not noted otherwise, and titrated with a standard solution of I₂ in 0.5 M LiCl in THF and diluted with THF to the indicated concentration. ZnBr₂ and ZnCl₂ were dried at 140° C. in high vacuum for 30 min and then dissolved in dry THF.

### General Regulation 1 (AV1): Nickel-Catalysed Chemical Reaction

The solution of the nickel catalyst was prepared as follows: anhydrous nickel chloride (8.2 mg, 0.063 mmol), (EtO)₂P(O)H (34.5 mg, 0.25 mmol) and DMAP (30.5 mg, 0.25 mmol) were dissolved in a 25 mL Schlenk tube under argon in dry, degassed N-ethylpyrrolidinone (10.0 mL). In an annealed 25 mL flask, that was rinsed with argon, equipped with a magnetic stirrer flask and a septum, the respective aryl magnesium reagent in THF (1.20 mmol) was slowly added by cooling the solution of ZnBr₂ (0.67 mL of a 1.5 molar solution in THF, 1.00 mmol) and NEP (0.17 mL). To this solution, the electrophile (aryl halide or sulfonate, 1.0 mmol) was added, followed by the solution of the catalyst (0.08 mL). The final THF-NEP volume ratio was supposed to be about 8:1. The reaction mixture was stirred at the indicated temperature until the gas-chromatographic check of an aliquot showed the complete reaction of the reaction products. The reaction was subsequently quenched with a saturated solution of NH₄Cl, extracted with ether and the product was purified by column chromatography.

### 3-fluoro-4′-methoxy-1,1′-biphenyle (3a)

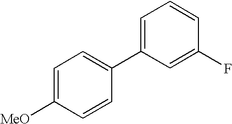

Preparation according to AV1. To the solution of ZnBr₂ (0.67 mL, 1.5 M in THF) and NEP (0.17 mL), 4-methoxyphenyl magnesium bromide (1.57 mL, 0.83 M in THF) was added dropwise and then the catalyst solution (0.08 mL) and 3-bromofluorobenzene (175 mg, 1.00 mmol) was added. This solution was stirred for 2 hours at room temperature. The conventional reprocessing and purification by column chromatography (pentane/ether19:1) yielded 3a as a white solid (174 mg, 86%).

mp: 67-67.5° C. (Lourak, M.; Vanderesse, R.; Fort, Y.; Caubere, P. J. Org. Chem. 1989, 54, 4844: 68° C.)

¹H NMR (CDCl₃, 300 MHz, 25° C.): δ=7.39 (d, J=8.9 Hz, 2H), 7.28-7.11 (m, 3H), 6.90-6.84 (m, 3H), 3.72 (s, 3H).

¹³C NMR (CDCl₃, 75 MHz, 25° C.): δ=163.2 (q, ¹J (C, F)=245 Hz), 159.5, 143.1 (q, ³J (C, F)=7.6 Hz), 132.4 (q, ⁴J (C, F)=2.1 Hz), 130.1 (q, ³J (C, F)=8.2 Hz), 128.1, 122.2 (q, ⁴J (C, F)=2.6 Hz), 114.5, 113.5 (q, ⁻²J (C, F)=21.7 Hz), 113.3 (q, ²J (C, F)=21.1 Hz), 55.3.

IR (KBr): 2963 (w), 2840 (w), 1610 (vs), 1589 (s), 1573 (m), 1522 (s), 1487 (s), 1447 (m), 1292 (s), 1264 (s), 1252 (s), 1189 (vs), 1162 (m), 1026 (m), 879 (m), 830 (vs), 782 (s).

MS (70 eV, EI), m/z (%): 209 (100, M⁺), 187 (50), 159 (54), 133 (24), 107 (10), 77 (13).

HRMS m/z: calculated for C₁₃H₁₁FO: 202.0794; found: 202.0790.

### ethyl-4′-methoxy-biphenyl-3-carboxylate (3b)

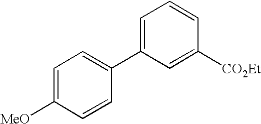

Preparation according to AV1. To the solution of ZnBr₂ (0.67 mL, 1.5 M in THF) and NEP (0.17 mL), 4-methoxyphenyl magnesium bromide (1.57 mL, 0.83 M in THF) was added dropwise and then the catalyst solution (0.08 mL) and ethyl-3-bromobenzoate (229 mg, 1.00 mmol) was added. This solution was stirred for 1 hour at room temperature. The conventional reprocessing and purification by column chromatography (pentane/ether 9:1) yielded 3b as a colourless oil (234 mg, 91%).

¹H NMR (CDCl₃, 300 MHz, 25° C.): δ=8.26 (s, 1H), 8.00-7.97 (m, 1H), 7.73-7.70 (m, 1H), 7.56 (d, J=8.8 Hz, 2H), 7.46 (t, J=7.7 Hz, 1H), 6.99 (d, J=8.7 Hz, 2H), 4.41 (q, J=7.1 Hz, 2H), 3.83 (s, 3H), 1.41 (t, J=7.1 Hz, 3H).

¹³C NMR (CDCl₃, 75 MHz, 25° C.): δ=166.5, 159.4, 140.9, 132.5, 130.9, 130.8, 128.6, 128.1, 127.6, 127.1, 114.2, 60.9, 55.2, 14.2.

IR (KBr): 2981 (w), 1717 (vs), 1610 (m), 1518 (s), 1439 (m), 1367 (w), 1300 (s), 1249 (vs), 1182 (m), 1109 (s), 1049 (m), 1030 (m), 834 (m), 758 (s), 574 (w).

MS (70 eV, EI), m/z (%): 256 (100, M⁺), 241 (9), 228 (11), 211 (20), 183 (10), 168 (6), 139 (12), 105 (3).

HRMS m/z: calculated for C₁₆H₁₆O₃: 256.1099; found: 256.1097.

### ethyl-4′-methoxy-biphenyl-4-carboxylate (3c)

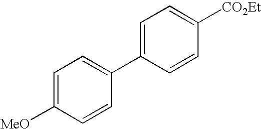

Preparation according to AV1. To the solution of ZnBr₂ (0.67 mL, 1.5 M in THF) and NEP (0.17 mL), 4-methoxyphenyl magnesium bromide (1.57 mL, 0.83 M in THF) was added dropwise and then the catalyst solution (0.08 mL) and ethyl-4-bromobenzoate (229 mg, 1.00 mmol) or ethyl-4-chlorobenzoate (185 mg, 1.00 mmol) was added. This solution was stirred for 1 hour at room temperature (48 hours for ethyl-4-chlorobenzoate). The conventional reprocessing and purification by column chromatography (pentane/ether 9:1) yielded 3c as a white solid (224 mg or 87% for the reaction with ethyl-4-bromobenzoate and 214 mg or 83% for ethyl-4-chlorobenzoate). The analytical data corresponds to literature (Nakao, Y.; Oda, T.; Sahoo, A. K.; Hiyama, T. J. Organomet. Chem. 2003, 687(2), 570).

mp: 100-101° C.

¹H NMR (CDCl₃, 300 MHz, 25° C.): δ=8.09 (d, J=8.7 Hz, 2H), 7.62-7.55 (m, 4H), 6.99 (d, J=8.7 Hz, 2H), 4.39 (q, J=7.1 Hz, 2H), 3.84 (s, 3H), 1.41 (t, J=7.1 Hz, 3H).

¹³C NMR (CDCl₃, 75 MHz, 25° C.): δ=166.5, 159.8, 145.0, 132.4, 130.0, 128.6, 128.3, 126.4, 114.3, 60.8, 55.3, 14.3.

### (4′-methoxy-[1,1-biphenyl]-4-yl)-(phenyl)-methanone (3d)

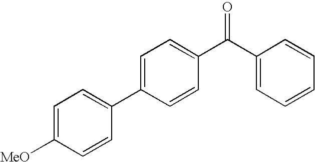

Preparation according to AV1. To the solution of ZnBr₂ (0.67 mL, 1.5 M in THF) and NEP (0.17 mL), 4-methoxyphenyl magnesium bromide (1.57 mL, 0.83 M in THF) was added dropwise and then the catalyst solution (0.08 mL) and 4-bromobenzophenone (261 mg, 1.00 mmol) was added. This solution was stirred for 3 hours at room temperature. The conventional reprocessing and purification by column chromatography (pentane/ether 19:1) yielded 3d as a white solid (210 mg, 73%). The analytical data corresponds to literature (Andrus, M. B.; Song, C. Org. Lett. 2001, 3, 3761).

mp: 167-168° C.

¹H NMR (CDCl₃, 600 MHz, 25° C.): δ=7.87 (d, J=8.1 Hz, 2H), 7.83 (d, J=8.3 Hz, 2H), 7.66 (d, J=8.3 Hz, 2H), 7.60-7.57 (m, 3H), 7.49 (t, J=7.6 Hz, 2H), 7.01 (d, J=8.8 Hz, 2H), 3.86 (s, 3H).

¹³C NMR (CDCl₃, 151 MHz, 25° C.): δ=196.3, 159.9, 144.8, 137.9, 135.6, 132.4, 132.2, 130.8, 129.9, 128.4, 128.3, 126.4, 114.4, 55.4.

IR (KBr): 1651 (vs), 1600 (vs), 1529 (w), 1446 (w), 1316 (m), 1288 (s), 1276 (s), 1256 (m), 1206 (vs), 1182 (w), 1033 (w), 939 (w), 829 (s), 794 (w), 697 (m).

MS (70 eV, EI), m/z (%): 288 (100, M⁺), 211 (76), 183 (6), 168 (8), 139 (8), 105 (11), 77 (10), 51 (1).

HRMS m/z: calculated for C₂₀H₁₆O₂: 288.1150; found: 288.1146.

### 3-(4-methoxyphenyl)-pyridine (3e)

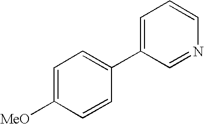

Preparation according to AV1. To the solution of ZnBr₂ (0.67 mL, 1.5 M in THF) and NEP (0.17 mL), 4-methoxyphenyl magnesium bromide (1.57 mL, 0.83 M in THF) was added dropwise and then the catalyst solution (0.08 mL) and 3-bromopyridine (159 mg, 1.00 mmol) or 3-chloropyridine (114 mg, 1.00 mmol) was added. This solution was stirred for 2 hours (12 hours for 3-chloropyridine) at room temperature. The conventional reprocessing and purification by column chromatography (pentane/ether 1:1) yielded 3e as a white solid (150 mg or 81% for 3-bromopyridine and 126 mg, 68% for 3-chloropyridine). The analytical data corresponds to literature (Cioffi, C. L.; Spencer, W. T.; Richards, J.; Herr, R. J. J. Org. Chem. 2004, 69, 2210). mp: 62-63° C.

¹H NMR (CDCl₃, 600 MHz, 25° C.): δ=8.81-8.80 (m, 1H), 8.53 (dd, J₁=4.8 Hz, J₂=1.6 Hz, 1H), 7.84-7.80 (m, 1H), 7.52 (d, J=8.8 Hz, 2H), 7.34-7.30 (m, 1H), 7.01 (d, J=8.8 Hz, 2H), 3.85 (s, 3H).

¹³C NMR (CDCl₃, 151 MHz, 25° C.): δ=159.7, 148.0, 147.9, 136.3, 133.8, 130.3, 128.2, 123.5, 114.6, 55.4.

IR (KBr): 2964 (w), 1608 (s), 1578 (w), 1564 (w), 1520 (s), 1478 (s), 1434 (m), 1283 (s), 1254 (vs), 1183 (s), 1030 (s), 838 (m), 803 (vs), 706 (m), 619 (w), 552 (w).

MS (70 eV, EI), m/z (%): 185 (100, M⁺), 170 (44), 142 (46), 115 (17), 89 (11), 63 (8).

HRMS m/z: calculated for C₁₂H₁₁NO: 185.0841; found: 185.0837.

### 6-(3-methoxyphenyl)-nicotinic acid methyl ester (3f)

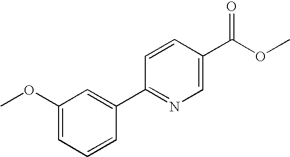

Preparation according to AV1. To the solution of ZnBr₂ (0.67 mL, 1.5 M in THF) and NEP (0.17 mL), 3-methoxy magnesium bromide (1.57 mL, 0.83 M in THF) was added dropwise and then the catalyst solution (0.08 mL in NEP) and 6-chloronicotinic acid methyl ester (172 mg, 1.00 mmol) was added. This solution was stirred for 24 hours at room temperature. The conventional reprocessing and purification by column chromatography (CH₂Cl₂-pentane 1:1) yielded 3f as a colourless solid (180 mg, 74%).

mp: 89.5-90° C.

¹H NMR (CDCl₃, 600 MHz, 25° C.): δ=9.24 (s, 1H), 8.31 (dd, J₁=8.3 Hz, J₂=1.9 Hz, 1H), 7.77 (d, J=8.3 Hz, 1H), 7.63-7.62 (m, 1H), 7.57 (d, J=7.9 Hz, 1H), 7.38 (t, J=8.1 Hz, 1H), 6.99 (dd, =8.1 Hz, J₂=2.4 Hz, 1H), 3.94 (s, 3H), 3.88 (s, 3H).

¹³C NMR (CDCl₃, 151 MHz, 25° C.): δ=165.9, 160.7, 160.2, 151.0, 139.7, 137.9, 129.9, 124.3, 120.0, 119.7, 116.1, 112.5, 55.4, 52.3.

IR (KBr): 3059 (w), 3013 (w), 2954 (m), 2925 (m), 1715 (vs), 1596 (vs), 1562 (m), 1480 (s), 1433 (s), 1288 (vs), 1267 (s), 1231 (s), 1117 (s), 1030 (s), 1021 (s).

MS (70 eV, EI), m/z (%): 243 (65, M⁺), 242 (100), 213 (38), 182 (9), 154 (10), 106 (11).

HRMS m/z: calculated for C₁₄H₁₃NO₃: 243.0895; found: 243.0867.

### 1-(3′-methoxybiphenyl-4-yl)-ethanone (3g)

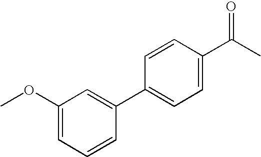

Preparation according to AV1. To the solution of ZnBr₂ (0.67 mL, 1.5 M in THF) and NEP (0.17 mL), 3-methoxy magnesium bromide (1.57 mL, 0.83 M in THF) was added dropwise and then the catalyst solution (0.08 mL) and 4-bromoacetonephenone (199 mg, 1.00 mmol) was added. This solution was stirred for 2.5 hours at room temperature. The conventional reprocessing and purification by column chromatography (CH₂Cl₂-pentane 1:1) yielded 3g as a yellow solid (175 mg, 77%).

mp: 35-36° C. Hatanaka, Y.; Goda, K.; Yoshinori, O.; Hiyama, T. Tetrahedron 1994, 50, 8301

¹H NMR (CDCl₃, 300 MHz, 25° C.): δ=8.00 (ddd, J₁=8.5 Hz, J₂=2.9 Hz, J₃=1.9 Hz, 2H), 7.65 (ddd, J₁=8.6 Hz, J₂=2.0 Hz, J₃=1.9 Hz, 2H), 7.38-7.31 (m, 1H), 7.20-7.17 (m, 1H), 7.13-7.12 (m, 1H), 6.94-6.90 (m, 1H), 3.85 (s, 3H), 2.61 (s, 3H).

¹³C NMR (CDCl₃, 75 MHz, 25° C.): δ=198.1, 160.4, 146.0, 141.8, 136.4, 130.4, 129.3, 127.7, 120.1, 113.9, 113.5, 55.8, 27.0.

MS (70 eV, EI), m/z (%): 226 (56, M⁺), 211 (100), 168 (14), 152 (11), 139 (21).

### 2-(3-pyridino)-benzophenone (3h)

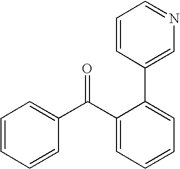

Preparation according to AV1. To the solution of ZnBr₂ (0.67 mL, 1.5 M in THF) and NEP (0.17 mL), 3-pyridyl magnesium bromide (Krasovskiy, A.; Knochel, P. Angew. Chem. Int. Ed. 2004, 3333) (1.57 mL, 0.83 M in THF) was added dropwise and then the catalyst solution (0.08 mL) and 2-bromobenzophenone (270 mg, 1.00 mmol) was added. This solution was stirred for 3 hours at 50° C. The conventional reprocessing and purification by column chromatography (pentane —CH₂Cl₂ 1:1) yielded 3h as a white solid (197 mg, 76%). The analytical data corresponds to literature (Edwards, M. L.; Stemerick, D. M; Diekema, K. A.; Dienerstein, R. J. J. Med. Chem. 1994, 37, 4357).

mp: 106-106.5° C.

¹H NMR (CDCl₃, 300 MHz, 25° C.): 8.56-8.52 (m, 1H), 8.44-8.40 (m, 1H), 7.72-7.10 (m, 11H).

¹³C NMR (CDCl₃, 75 MHz, 25° C.): 198.3, 149.8, 148.8, 139.5, 137.9, 137.6, 136.6, 136.3, 133.6, 131.1, 130.6, 130.3, 129.5, 128.7, 128.2, 123.3.

MS (70 eV, EI), m/z (%): 77 (27), 105 (25), 127 (20), 182 (36), 230 (100), 231 (26), 259 (19, M⁺).

### 5-(3-fluorophenyl)-pyrimidine (3l)

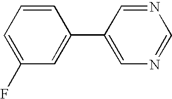

Preparation according to AV1. To the solution of ZnBr₂ (0.67 mL, 1.5 M in THF) and NEP (0.17 mL), 3-fluorophenyl magnesium bromide (1.57 mL, 0.83 M in THF) was added dropwise and then the catalyst solution (0.08 mL) and 5-bromopyrimidine (159 mg, 1.00 mmol) was added. This solution was stirred for 1 hour at room temperature. The conventional reprocessing and purification by column chromatography (pentane-Et₂O) yielded 3l as a white solid (143 mg, 82%).

mp: 63-63.5° C.

¹H NMR (CDCl₃, 300 MHz, 25° C.): 9.13 (s, 1H), 8.85 (s, 2H), 7.44-7.23 (m, 1H), 7.29-7.26 (m, 1H), 7.22-7.17 (m, 1H), 7.10-7.03 (m, 1H).

¹³C NMR (CDCl₃, 75 MHz, 25° C.): 163.7 (d, J=248 Hz), 158.3, 155.2, 136.8 (d, J=7.9 Hz), 133.5, 131.5 (d, J=8.5 Hz), 123.0, 116.3 (d, J=21.1 Hz), 114.3 (d, J=21.1 Hz).

IR (KBr): 2239 (w), 1591 (s), 1416 (s), 909 (vs), 734 (vs). MS (70 eV, EI), m/z (%): 94 (12), 105 (25), 120 (100), 173 (21), 174 (96, M⁺).

HRMS m/z: calculated for C₁₀H₇N₂F: 174.0593; found: 174.0577.

### 4-pyrimidine-5-yl-benzoic acid ethyl ester (3j)

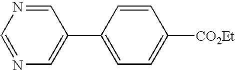

Preparation according to AV1. To the solution of ZnBr₂ (0.67 mL, 1.5 M in THF) and NEP (0.17 mL), cold 4-carboethoxyphenyl magnesium bromide (prepared by iodine-magnesium displacement from 4-iodinebenzoate and iPrMgCl-LiCl (Krasovskiy, A.; Knochel, P. Angew. Chem. Int. Ed. 2004, 3333) at −40° C. over a period of 30 minutes) (1.57 mL, 0.83 M in THF) was added dropwise and then the catalyst solution (0.08 mL) and 5-bromopyrimidine (159 mg, 1.00 mmol) was added. This solution was stirred for 24 hours at room temperature. The conventional reprocessing and purification by column chromatography (CH₂Cl₂-pentane) yielded 3j as light yellow crystals (137 mg, 60%). The analytical data corresponds to literature (Kano, S.; Yuasa, Y.; Shibuya, S.; Hibino, S. Heterocycles, 1982, 19, 1079).

mp: 118-119° C.

¹H NMR (CDCl₃, 600 MHz, 25° C.): δ=9.25 (s, 1H), 8.99 (s, 2H), 8.19 (m, 2H), 7.66 (m, 2H), 4.42 (q, J=7.2 Hz, 2H), 1.43 (t, J=7.2 Hz, 3H).

¹³C NMR (CDCl₃, 150 MHz, 25° C.): δ=164.9, 157.2, 157.0, 154.1, 154.0, 137.5, 132.4, 130.0, 129.6, 125.9, 60.3, 13.3.

MS (70 eV, EI), m/z (%): 228 (21, M⁺), 200 (33), 183 (100), 128 (40), 101 (32).

### 8-(1-naphthyl)-quinoline (3k)

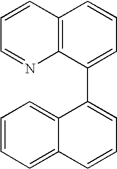

Preparation according to AV1. To the solution of ZnBr₂ (0.67 mL, 1.5 M in THF) and NEP (0.17 mL), 1-naphthyl magnesium bromide (1.57 mL, 0.83 M in THF) was added dropwise and then the catalyst solution (0.08 mL) and 8-quinolylnonaflate (Subramanian, L. R.; Garcia Martinez, A.; Herrera Fernandez, A.; Martinez Alvarez, R. Synthesis, 1984, 6, 481) (427 mg, 1.00 mmol) was added. This solution was stirred for 24 hours at room temperature. The conventional reprocessing and purification by column chromatography (CH₂Cl₂-pentane) yielded 3k as a white solid (224 mg, 88%).

mp: 163-164° C.

¹H NMR (CDCl₃, 300 MHz, 25° C.): 8.76-8.74 (m, 1H); 8.16-8.13 (m, 1H); 7.89-7.82 (m, 3H); 7.69-7.66 (m, 1H), 7.60-7.46 (m, 3H), 7.41-7.18 (m, 4H).

¹³C NMR (CDCl₃, 75 MHz, 25° C.): δ=150.9, 147.7, 140.6, 138.5, 136.6, 134.1, 133.3, 132.0, 128.9, 128.7, 128.5, 128.4, 128.3, 127.1, 126.6, 126.1, 126.0, 125.8, 121.5.

IR: (KBr) (cm⁻¹): 3042 (w), 1593 (w), 1492 (s), 829 (s), 797 (vs), 782 (vs), 773 (vs).

MS (70 eV, EI), m/z (%): 127 (9), 226 (9), 252 (14), 254 (100), 255 (47, M⁺).

HRMS m/z: calculated for C₁₉H₁₃N, 255.1048; found: 255.1020.

### 3-(1-methyl-1H-pyrrole-2-yl)-pyridine (3l)

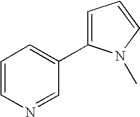

Preparation according to AV1. To the solution of ZnBr₂ (0.67 mL, 1.5 M in THF) and NEP (0.17 mL), 1-methyl-2-pyrryllithium (Brittain, J. M.; Jones, R. A.; Argues, J. S.; Saliente, T. A. Synth. Comm. 1982, 12, 231) (2.4 mL, 0.5 M in THF) was added dropwise and then the catalyst solution (0.08 mL) and 3-bromopyridine (158 mg, 1.00 mmol) was added. This solution was stirred for 22 hours at 70° C. The conventional reprocessing and purification by column chromatography (Et₂O—CH₂Cl₂ 1:1) yielded 3l as yellow oil (98 mg, 62%). The analytical data corresponds to literature (Baxendale, I.; Brusotti, M.; Ley, S. J. Chem. Soc. Perkin 1, 2002, 143).

¹H NMR (CDCl₃, 300 MHz, 25° C.): δ=8.66 (d, J=1.8 Hz, 1H), 8.50 (dd, =4.8 Hz, J₂=1.6 Hz, 1H), 7.70 (ddd, J₁=7.9 Hz, J₂=1.8 Hz, J₃=1.6 Hz, 1H), 7.31 (ddd, =7.9 Hz, J₂=4.8 Hz, J₃=0.8 Hz, 1H), 6.74 (dd, J₁=2.5 Hz, J₂=1.9 Hz, 1H), 6.27 (dd, J₁=3.6 Hz, J₂=1.9 Hz, 1H), 6.20 (dd, J₁=3.6 Hz, J₂=2.8 Hz, 1H), 3.65 (s, 3H).

¹³C NMR (CDCl₃, 75 MHz, 25° C.): δ=149.3, 147.8, 136.1, 131.1, 129.8, 125.2, 123.7, 110.3, 108.7, 35.5.

MS (70 eV, EI), m/z (%): 158 (100, M⁺), 143 (7), 130 (19), 116 (6), 89 (5).

### 1-(3′-trifluoromethylbiphenyl-4-yl)-ethanone (3m)

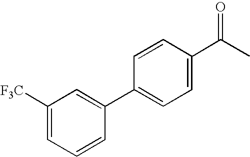

Preparation according to AV1. To the solution of ZnBr₂ (0.67 mL, 1.5 M in THF) and NEP (0.17 mL), 3-trifluoromethylphenyl magnesium bromide (Krasovskiy, A.; Knochel, P. Angew. Chem. Int. Ed. 2004, 3333) (1.57 mL, 0.5 M in THF) was added dropwise and then the catalyst solution (0.08 mL) and 4-bromoacetonephenone (199 mg, 1.00 mmol) was added. This solution was stirred for 18 hours at room temperature. The conventional reprocessing and purification by column chromatography (CH₂Cl₂-petane 1:1) yielded 3m as colourless oil (180 mg, 68%). The analytical data corresponds to literature (Solodenko, W.; Schon, U.; Messinger, J.; Glinschert, A.; Kirschning, A. Synlett 2004, 10, 1699).

¹H NMR (CDCl₃, 300 MHz, 25° C.): δ=8.06 (ddd, J₁=8.6 Hz, J₂=2.4 Hz, J₃=2.0 Hz, 2H), 7.87-7.78 (m, 2H), 7.73-7.57 (m, 4H), 2.65 (s, 3H).

¹³C NMR (CDCl₃, 75 MHz, 25° C.): δ=197.9, 144.6, 141.1, 136.9, 132.2, 132.0, 131.6, 130.9, 130.2, 129.9, 129.5, 127.4, 125.3, 124.4, 27.1.

MS (70 eV, EI), m/z (%): 264 (35, M⁺), 249 (100), 221 (6), 201 (34), 152 (21).

### ethyl 4-(1,3-benzodioxol-5-yl)benzoate (3n)

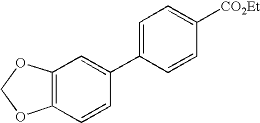

Preparation according to AV1. To the solution of ZnBr₂ (0.67 mL, 1.5 M in THF) and NEP (0.17 mL), 1,3-benzodioxol-5-yl magnesium bromide (1.57 mL, 0.83 M in THF) was added dropwise and then the catalyst solution (0.08 mL) and 4-ethyl-4-bromobenzoate (229 mg, 1.00 mmol) was added. This solution was stirred for 5 hours at room temperature. The conventional reprocessing and purification by column chromatography (pentane ether 1:1) yielded 3n as a white solid (253 mg, 94%).

mp: 92.5-93.5° C.

¹H NMR (CDCl₃, 300 MHz, 25° C.): δ=8.07 (d, J=8.7 Hz, 2H), 7.56 (d, J=8.7 Hz, 2H), 7.11-7.07 (m, 2H), 6.89 (d, J=8.6 Hz, 1H), 5.00 (s, 2H), 4.39 (q, J=7.1 Hz, 2H), 1.40 (d, J=7.2 Hz, 3H).

¹³C NMR (CDCl₃, 75 MHz, 25° C.): δ=166.4, 148.3, 147.7, 145.1, 134.3, 130.0, 128.8, 126.6, 121.0, 108.6, 107.6, 101.3, 60.9, 14.3.

IR (KBr): 2904 (w), 1707 (vs), 1606 (m), 1522 (w), 1503 (m), 1486 (s), 1410 (s), 1274 (vs), 1256 (s), 1235 (m), 1182 (s), 1107 (s), 1036 (s), 932 (m), 858 (m), 772 (s), 702 (w).

MS (70 eV, EI), m/z (%): 270 (100, M⁺), 242 (32), 225 (70), 139 (40), 112 (5), 63 (2).

HRMS m/z: calcd. for O₁₆H₁₄O₄: 270.0892; found: 270.0888.

### 3-(1,3-benzodioxol-5-yl)pyridine (3o)

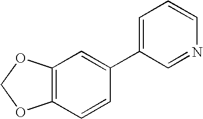

Preparation according to AV1. To the solution of ZnBr₂ (0.67 mL, 1.5 M in THF) and NEP (0.17 mL), 1,3-benzodioxol-5-yl magnesium bromide (1.57 mL, 0.83 M in THF) was added dropwise and then the catalyst solution (0.08 mL) and 3-bromopyridine (158 mg, 1.00 mmol) was added. This solution was stirred for 5 hours at room temperature. The conventional reprocessing and purification by column chromatography yielded 3o as a white solid (165 mg, 83%).

mp: 92-92.5° C.

¹H NMR (CDCl₃, 300 MHz, 25° C.): δ=8.76 (d, J=1.9 Hz, 1H), 8.54-8.51 (m, 1H), 7.78-7.74 (m, 1H), 7.32-7.27 (m, 1H), 7.04-7.00 (m, 2H), 6.90-6.87 (m, 1H), 5.99 (s, 2H).

¹³C NMR (CDCl₃, 75 MHz, 25° C.): δ=148.4, 148.1, 148.0, 147.7, 136.3, 133.9, 131.9, 123.4, 120.8, 108.8, 107.5, 101.3.

IR (KBr): 2912 (w), 1512 (s), 1479 (vs), 1420 (s), 1294 (w), 1266 (m), 1238 (s), 1111 (w), 1037 (s), 931 (m), 806 (s), 706 (m).

MS (70 eV, EI), m/z (%): 199 (100, M⁺), 140 (10), 114 (11), 88 (4), 63 (3).

HRMS m/z: calcd. for C₁₂H₉NO₂: 199.0633; found: 199.0602.

### 1-(3,4-methylenedioxyphenyl)-naphthalene (3p)

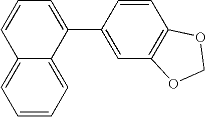

Preparation according to AV1. To the solution of ZnBr₂ or solution of ZnCl₂ (0.67 mL, 1.5 M in THF) and NEP (0.17 mL), 1-naphthyl magnesium bromide (1.57 mL, 0.83 M in THF) was added dropwise and then the catalyst solution (0.08 mL) and 3,4-methylenedioxylphenyltriflate (Echavarren, A. M.; Stifle, J. K. J. Am. Chem. Soc. 1987, 109, 5478) (270 mg, 1.00 mmol) was added. This solution was stirred for 24 hours at room temperature. The conventional reprocessing and purification by column chromatography (pentane-Et₂O 9:1) yielded 3p as colourless oil (196 mg, 79%, the reaction with ZnCl₂ gave 77%). The analytical data corresponds to literature (Shimada, S.; Yamazaki, 0.; Toshifumi, T.; Rao, M.; Suzuki, Y.; Tanaka, M. Angew. Chem. Int. Ed. 2003, 42, 1845).

¹H NMR (CDCl₃, 300 MHz, 25° C.): S=7.99-7.85 (m, 3H), 7.55-7.41 (m, 4H), 7.02-6.94 (m, 3H), 6.06 (s, 2H).

¹³C NMR (CDCl₃, 75 MHz, 25° C.): δ=147.9, 174.3, 140.2, 135.1, 134.2, 132.2, 128.7, 128.0, 127.3, 126.4, 126.2, 125.8, 123.8, 111.1, 108.6, 101.5.

MS (70 eV, EI), m/z (%): 248 (100, M⁺), 217 (19), 208 (10), 189 (52), 94 (20).

General Regulation 2 (AV2): Nickel-Catalysed Chemical Reaction

0.93 mL of a 1.5 M solution of ZnBr₂/THF (1.4 mmol) and 0.25 mL N-ethylpyrrolidinone (NEP) are presented in an annealed Schlenk tube under argon. 1.2 mmol of aryl magnesium halide in a solution of THF is added dropwise. Subsequently, 1.0 mmol aryl halide, then 0.025 mL of a 0.08 M solution of 4-dimethylaminopyridine (DMAP)-(EtO)₂P(O)H in THF (0.2 mole percent) and 0.025 mL of a 0.02 M solution of NiCl₂ in NEP (0.05 mole percent Ni) are added.

After an appropriate time, the reaction is stopped with a saturated solution of NH₄Cl. The mixture is extracted with Et₂O, the united organic phases are dried over MgSO₄ and concentrated in vacuum. The residue is purified by chromatography (SiO₂).

All compounds listed in table 1 below were synthesized according to the general regulation 2.

TABLE 1

| no. | nucleophile | electrophile | product | yield |
|---|---|---|---|---|
| 1 | 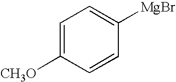 | 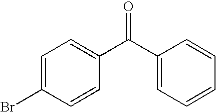 | 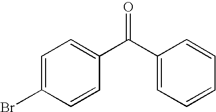 RT, 3 h | 73% |
| 2 | 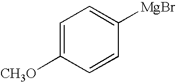 | 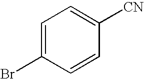 | 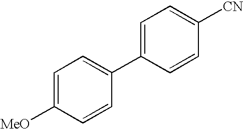 50° C., 72 h | 54% |
| 3 | 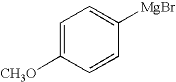 | 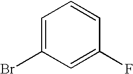 | 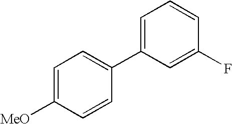 RT, 2 h | 79% |
| 4 | 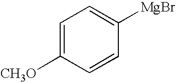 | 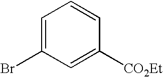 | 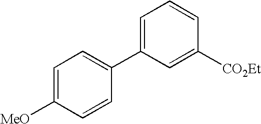 RT, 6 h | 91% |
| 5 | 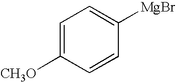 | 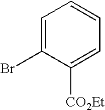 | 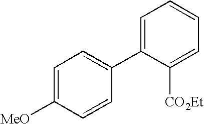 RT, 6 h | 52% |
| 6 | 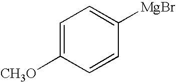 | 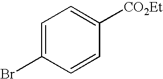 | 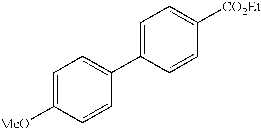 RT, 6 h | 87% |
| 7 | 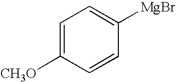 | 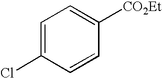 | 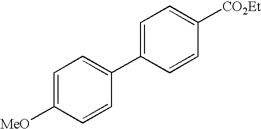 50° C., 48 h | 83% |
| 8 | 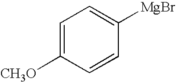 | 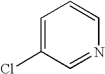 | 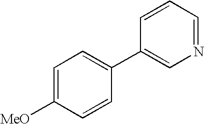 RT, 12 h | 68% |
| 9 | 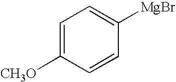 | 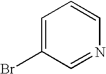 | 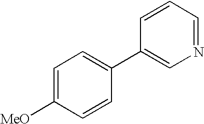 RT, 2 h | 81% |
| 10 | 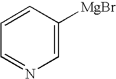 | 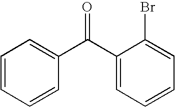 | 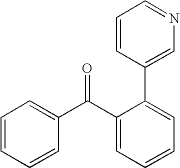 50° C., 3 h | 76% |
| 11 | 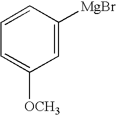 | 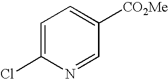 | 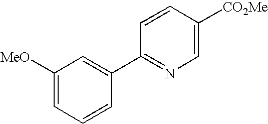 RT, 24 h | 74% |
| 12 | 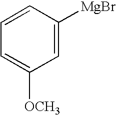 | 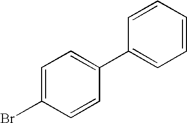 | 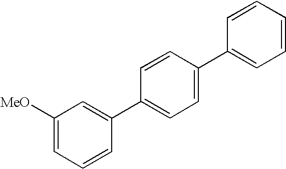 RT, 4 h | 77% |
| 13 | 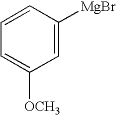 | 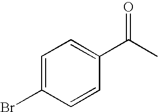 | 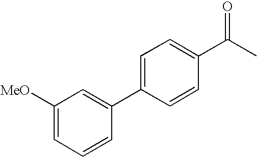 RT, 2.5 h | 77% |
| 14 | 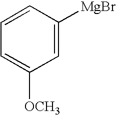 | 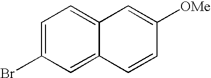 | 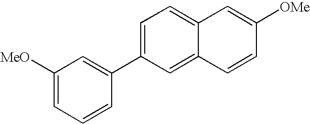 RT, 8 h | 80% |
| 15 | 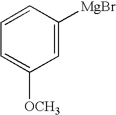 | 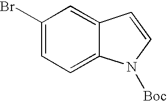 | 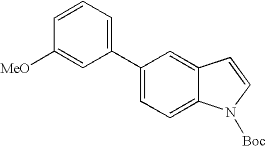 RT, 24 h | 60% |
| 16 | 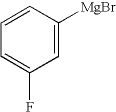 | 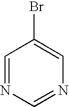 | 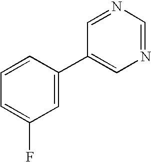 RT, 1 h | 82% |
| 17 | 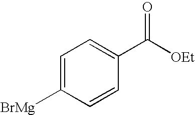 | 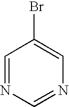 | 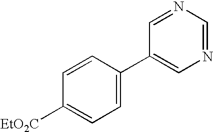 50° C., 24 h | 60% |
| 18 | 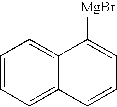 | 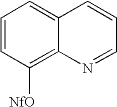 | 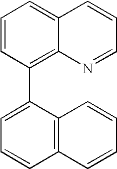 RT, 24 h | 88% |
| 19 | 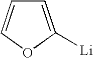 | 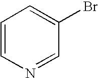 | 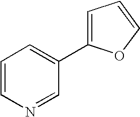 110° C., 6 h | 59% |
| 20 | 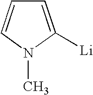 | 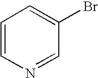 | 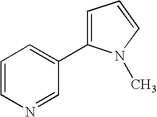 70° C., 22 h | 62% |
| 21 | 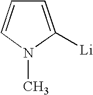 | 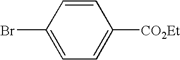 | 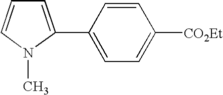 70° C., 22 h | 61% |
| 22 | 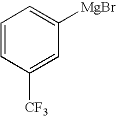 | 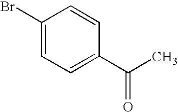 | 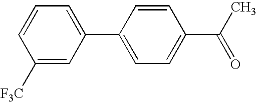 RT, 18 h | 68% |
| 23 | 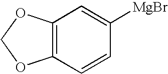 | 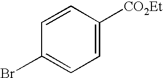 | 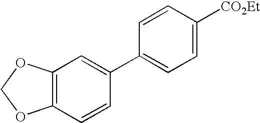 RT, 5 h | 94% |
| 24 | 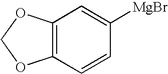 | 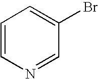 | 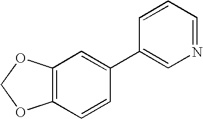 RT, 5 h | 83% |
| 25 | 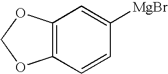 | 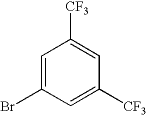 | 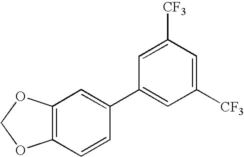 RT, 5 h | 83% |
| 26 | 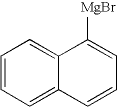 | 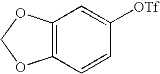 | 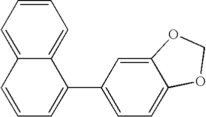 RT, 24 h | 79% (77% with ZnCl₂) |
| 27 | 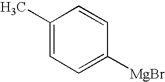 | 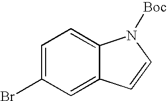 | 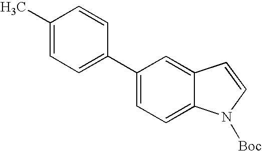 RT, 8 h | 75% |
| 28 | 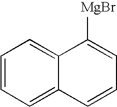 | 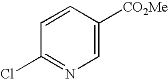 | 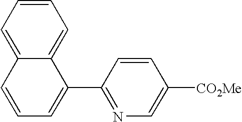 50° C., 24 h | 73% |
| 29 | 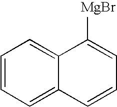 | 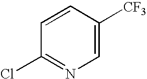 | 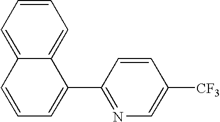 95° C., 2 h | 82% |
| 30 | 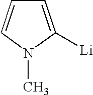 | 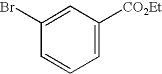 | 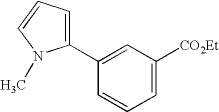 70° C., 22 h | 55% |
| 31 | 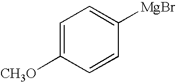 | 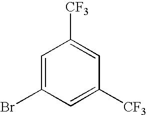 | 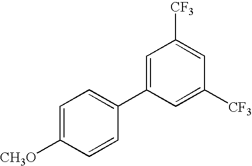 RT, 2 h | 53% |
| 32 | 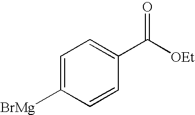 | 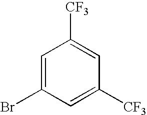 | 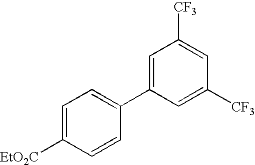 110° C., 5 h | 55% |

### General Regulation 3 (AV3): Iron-Catalysed Chemical Reaction

Aryl magnesium bromide (1.3 mmol, in THF) is presented in an annealed Schlenk tube, a solution of ZnBr₂ (1.3 mmol, 0.65 mL, 2.0 M in NMP) is added and the mixture is stirred for 15 min at room temperature (RT). Subsequently, NMP (0.5 mL), Fe(DBM)₃ (5 mole percent, 36 mg) and aryl halide (1.0 mmol) are added and the reaction mixture is stirred for an appropriate time at 110° C. Subsequently, the reaction is stopped by adding sat. NH₄Cl(aq.) and extracted with EtOAc (3×40 mL). The united organic phases are washed with sat. NaCl(aq.) (50 ml), dried over Na₂SO₄, filtrated and the solvent is removed by destillation under reduced pressure. The purification by column chromatography (DCM) yielded the desired product.

All products from table 2 were synthesized according to AV3, except for entry 10, which was synthesized according to the following instruction.

### Preparation of 4′-cyano-biphenyl-4-carboxylic acid ethyl ester

4-Iodineethylbenzoate (1.3 mmol, 359 mg) is presented in an annealed Schlenk tube, at −20° C. a solution of iPrMgCl (1.35 mmol, 1.38 mL, 0.98 M in THF) is added and the mixture is stirred for 30 min. Subsequently, a solution of ZnBr₂ (1.3 mmol, 0.65 mL, 2.0 M in NMP) is added and stirred for 15 min at room temperature. Now, NMP (0.5 mL), Fe(DBM)₃ (5 mole percent, 36 mg) and 4-bromobenzonitrile (1.0 mmol, 182 mg) are added and the reaction mixture is stirred for 6 hours at 110° C. Then the reaction is stopped by adding sat. NH₄Cl(aq.) and extracted with EtOAc (3×40 mL). The united organic phases are washed with sat. NaCl(aq.) (50 mL), dried over Na₂SO₄, filtrated and the solvent is removed by destillation under reduced pressure. The purification by column chromatography (pentane-diethylether) yielded the desired product as a colourless solid (169 mg, 67%).

TABLE 2
Preparation of biaryls from aryl-zinc reagents and
aryl halides by Fe-catalysed cross coupling:

| | aryl-zinc | | | | reaction |
|---|---|---|---|---|---|
| entry | reagent | electrophile | product | yield | time |
| 1 | 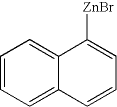 | 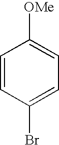 | 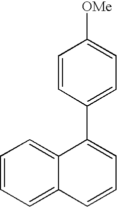 | 79% | 24 h |
| 2 | 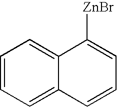 | 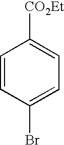 | 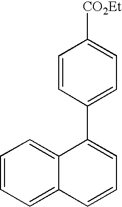 | 82% | 36 h |
| 3 | 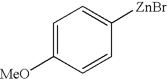 | 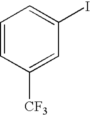 | 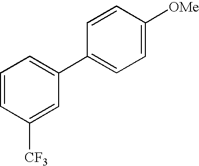 | 56% | 20 h |
| 4 | 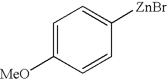 | 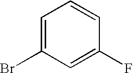 | 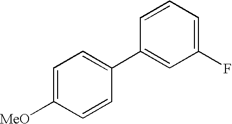 | 83% | 24 h |
| 5 | 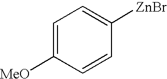 | 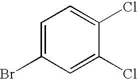 | 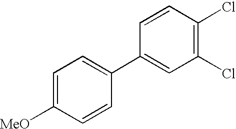 | 63% | 4 h |
| 6 | 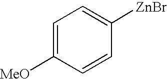 | 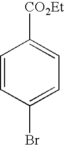 | 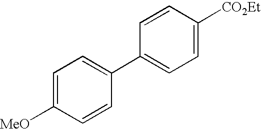 | 80% | 3 h |
| 7 | 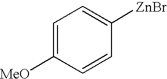 | 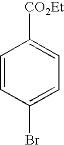 | 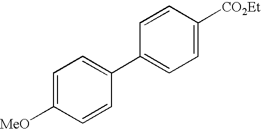 | 78% 10 mM scale | 3 h |
| 8 | 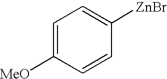 | 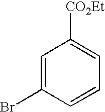 | 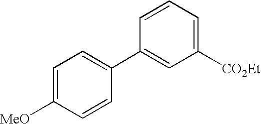 | 73% | 3 h |
| 9 | 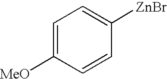 | 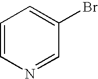 | 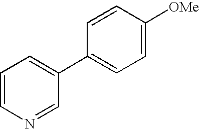 | 58% | 16 h |
| 10 | 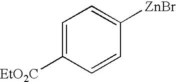 | 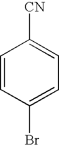 | 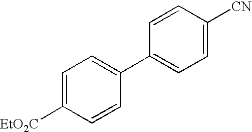 | 56% | 24 h |
| 11 | 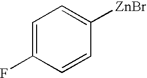 | 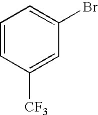 | 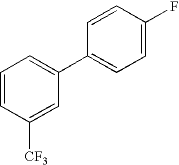 | 55% | 20 h |
| 12 | 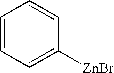 | 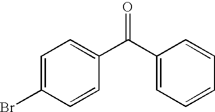 | 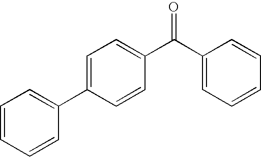 | 60% | 5 h |

### REFERENCES

[1] (a) Metal catalyzed Cross-Coupling Reactions; F. Diederich; P. J. Stang Eds. Wiley-VCH, Weinheim, 1998; (b) J. Tsuji, Transition Metal Reagents and Catalysts: Innovations in Organic Synthesis; Wiley: Chichester, 1995; (c) Cross-Coupling Reactions. A Practical Guide; N. Miyaura Ed. Top. Curr. Chem. 2002, 219, Springer-Verlag Berlin, Heidelberg, N.Y.
[2] Handbook of organopalladium chemistry for organic synthesis, Ed. E. Negishi, Wiley-Interscience, New York, 2002.
[3] (a) C. Dai, G. C. Fu, J. Am. Chem. Soc. 2001, 123, 2719; (b) A. F. Littke, G. C. Fu, J. Am. Chem. Soc. 2001, 123, 6989; (c) A. F. Littke, L. Schwarz, G. C. Fu, J. Am. Chem. Soc. 2002, 124, 6343; (d) A. F. Littke, G. C. Fu, Angew. Chem. 2002, 114, 4350; Angew. Chem. Int. Ed. 2002, 41, 4176; (e) I. D. Hills, M. R. Netherton, G. C. Fu, Angew. Chem. 2003, 115, 5927; Angew. Chem. Int. Ed. 2003, 42, 5749; (g) A. C. Frisch, A. Zapf, O. Briel, B. Kayser, N. Shaikh, M. Beller, J. Mol. Catalysis. A 2004, 214, 231; (h) A. Tewari, M. Hein, A. Zapf, M. Beller, Synthesis 2004, 935; (i) F. Rataboul, A. Zapf, R. Jackstell, S. Harkal, T. Riermeier, A. Monsees, U. Dingerdissen, M. Beller, Chem. Eur. J. 2004, 10, 2983.

[4] (a) M. Tamura, J. K. Kochi, J. Am. Chem. Soc. 1971, 93, 1487; (b) M. Tamura, J. K. Kochi, Synthesis 1971, 93, 303; (c) M. Tamura, J. K. Kochi, J. Organomet. Chem. 1971, 31, 289; (d) M. Tamura, J. K. Kochi, Bull. Chem. Soc. Jpn. 1971, 44, 3063; (e) M. Tamura, J. K. Kochi, Synthesis 1971, 303; (f) J. K. Kochi, Acc. Chem. Res. 1974, 7, 351; (g) S, Neumann, J. K. Kochi, J. Org. Chem. 1975, 40, 599; (h) R. S. Smith, J. K. Kochi, J. Org. Chem. 1976, 41, 502; (i) Furthermore: G. Molander, B. Rahn, D. C. Shubert, S. E. Bonde, Tetrahedron Lett. 1983, 24, 5449.
[5] (a) G. Cahiez, S. Marquais, Pure Appl. Chem. 1996, 68, 669; (b) G. Cahiez, S. Marquais, Tetrahedron Lett. 1996, 37, 1773; (c) G. Cahiez, H. Avedissian, Synthesis 1998, 1199; (d) K. Shinokubo, K. Oshima, Eur. J. Org. Chem. 2004, 2081; (e) M. A. Fakhfakh, X. Franck, R. Hocquemiller, B. Figadère, J. Organomet. Chem. 2001, 624, 131; (f) M. Hocek, H. Dvoráková, J. Org. Chem. 2003, 68, 5773; (g) B. Holzer, R. W. Hoffmann, Chem. Comm. 2003, 732; (h) W. Dohle, F. Kopp, G. Cahiez, P. Knochel, Synlett 2001, 1901; (i) M. Hojo, Y. Murakami, H. Aihara, R. Sakuragi, Y. Baba, A. Hosomi, Angew. Chem. 2001, 113, 641; Angew. Chem. Int. Ed. 2001, 40, 621; (j) M. Nakamura, A. Hirai, E. Nakamura, J. Am. Chem. Soc. 2001, 122, 978; (k) E. Alvarez, T. Cuvigny, C. H. du Penhoat, M. Julia, Tetrahedron 1998, 44, 119; (I) V. Finandanese, G. Marchese, V. Martina, L. Ronzini, Tetrahedron Lett. 1984, 25, 4805.
[6] (a) A. Furstner, A. Leitner, M. Mendez, H. Krause, J. Am. Chem. Soc. 2002, 124, 13856; (b) A. Fürstner, A. Leitner, Angew. Chem. 2002, 114, 632; Angew. Chem. Int. Ed. 2002, 41, 2003, 42, 308; (c) A. Fürstner, A. Leitner, Angew. Chem. 2003, 115, 320; Angew. Chem. Int. Ed. 2003, 42, 308; (d) G. Seidel, D. Laurich, A. Fürstner, J. Org. Chem. 2004, 69, 3950; (e) B. Scheiper, M. Bonnekessel, H. Krause, A. Fürstner, J. Org. Chem. 2004, 69, 3943; (f) A. Fürstner, D. De Souza, L. Parra-Rapado, J. T. Jensen, Angew. Chem. 2003, 115, 5516; Angew. Chem. Int. Ed. 2003, 42, 5355; (g) M. Nakamura, K. Matsuo, S. Ito, E. Nakamura, J. Am. Chem. Soc. 2004, 126, 3686; (h) T. Nagano, T. Hayashi, Org. Lett. 2004, 6, 1297; (i) R. Martin, A. Fürstner, Angew. Chem. 2004, 116, 4045; Angew. Chem. Int. Ed. 2004, 43, 3955.

1. A method for preparing a compound represented by the general formula (3)
R¹—Ar¹—Ar²—R² (3)
by reacting a compound represented by the general formula (1)
R¹—Ar¹—ZnY (1)
with a compound represented by the general formula (2)
R²—Ar²—X (2)
by effect of a Ni or Fe catalyst in a solvent, wherein
X may be a leaving group suitable for a nucleophilic substitution;
Y may be Cl, Br, I, R¹COO, ½ SO₄, NO₃, R¹SO₃;
R¹ and R² may each represent independently from one another one or more substituents of H; substituted or unsubstituted aryl or heteroaryl, containing one or more heteroatoms; straight-chain, branched or cyclic, substituted or unsubstituted alkyl, alkenyl, alkynyl; or derivatives thereof;
Ar¹ and Ar² may each represent independently from one another an aryl, condensed aryl, heteroaryl or condensed heteroaryl, containing one or more heteroatoms; an alkenyl or alkynyl; or derivatives thereof;

2. (canceled)

3. (canceled)

4. (canceled)

5. (canceled)

6. (canceled)

7. (canceled)

8. (canceled)

9. (canceled)

10. (canceled)

11. The method of claim 1, wherein the catalyst comprises a Ni(II) complex, a Ni(II) salt or a Ni(0) complex, or a reduced form of a Ni salt or complex.

12. The method of claim 11, wherein the catalyst comprises a Ni(II) salt besides DMAP and/or (EtO)₂P(O)H.

13. The method of claim 1, wherein the reaction by effect of a Ni catalyst is effected at a temperature between 0° C. and 150° C.

14. The method of claim 1, wherein the reaction by effect of a Ni catalyst is effected at a temperature between 10° C. and 120° C.

15. The method of claim 1, wherein the reaction by effect of a Ni catalyst is effected at a temperature between 20° C. and 100° C.

16. The method of claim 1, wherein the reaction by effect of a Ni catalyst is effected at a temperature between 25° C. and 80° C.

17. The method of claim 1, wherein the catalyst comprises a Fe(III) complex, a Fe(III) salt, a Fe(II) complex, a Fe(II) salt or a reduced form of a Fe salt or complex.

18. The method of claim 17, wherein the catalyst comprises Fe(acac)₃ or Fe(DBM)₃.

19. The method of claim 1, wherein the catalyst represents a complex with aza-heterocycles, polyaza-heterocycles and/or (R^{a}O)₂P(O)H, wherein R^{a} is a straight-chain, branched or cyclic, substituted or unsubstituted alkyl, as ligands.

20. The method of claim 1, wherein X is I, Br, CI, OTf, N₂⁺, OSO₂R^{S}, OP(O)(OR^{S})₂, wherein R^{S} is a straight-chain, branched or cyclic, substituted or unsubstituted alkyl, condensed aryl, substituted or unsubstituted aryl or heteroaryl.

21. The method of claim 1, wherein the compound (1) is added in a molar ratio of 0.2-5 with regard to the molar amount of compound (2).

22. The method of claim 1, wherein the compound (1) is added in a molar ratio of 1-3 with regard to the molar amount of compound (2).

23. The method of claim 1, wherein the compound (1) is added in a molar ratio of 1.1-2.5 with regard to the molar amount of compound (2).

24. The method of claim 1, wherein R¹ and R² may each be independently from one another a substituted or unsubstituted C₄-C₂₄ aryl or C₃-C₂₄ heteroaryl, containing one or more heteroatoms such as B, O, N, S, Se, P; a straight-chain or branched, substituted or unsubstituted O₁—O₂₀ alkyl, C₂-C₂₀ alkenyl, O₂—O₂₀ alkynyl; or a substituted or unsubstituted C₃-C₂₀ cycloalkyl; or derivatives thereof.

25. The method of claim 1, wherein the catalyst is used in a molecular ratio of 0.00001 to 10 mole percent with regard to the compound represented by the formula (1) or (2).

26. The method of claim 1, wherein the catalyst is used in a molecular ratio of 0.001 to 1 mole percent with regard to the compound represented by the formula (1) or (2).

27. The method of claim 1, wherein the catalyst is used in a molecular ratio of 0.02 to 0.2 mole percent with regard to the compound represented by the formula (1) or (2).

28. The method of claim 1, wherein a polar solvent or solvent mixture is used as solvent.

29. The method of claim 1, wherein the solvent is an etherial solvent, a dipolar, aprotic solvent or their solvent mixtures.

30. The method of claim 1, wherein the solvent is selected from the group comprising THF, DME, NEP, DMPU, DMI, NMP, DMAC and their mixtures,
